(19) United States
(12) Patent Application Publication
(10) Pub. No.: US 2023/0276862 A1
(43) Pub. Date: Sep. 7, 2023

(54) ORAL DISPENSER FOR LIQUIDS, IN PARTICULAR FOR LIQUIDS CONTAINING NICOTINE OR CANNABIS

(71) Applicant: Aptar Radolfzell GmbH, Radolfzell (DE)

(72) Inventor: Jürgen GREINER-PERTH, Gottmadingen (DE)

(21) Appl. No.: 18/004,330

(22) PCT Filed: Jul. 1, 2021
(86) PCT No.: PCT/EP2021/068263

§ 371 Date: Jan. 5, 2023

(30) Foreign Application Priority Data
Jul. 7, 2020 (EP) 20184586.4

(51) Int. Cl. A24F 42/60; A24F 42/20

(52) U.S. Cl. CPC A24F 42/60; A24F 42/20

(57) ABSTRACT

An oral dispenser for discharging a liquid in atomized form. This oral dispenser has a liquid reservoir of variable internal volume for receiving the liquid prior to the discharge, and a spring mechanism. When the spring mechanism is loaded, the liquid in the liquid reservoir is placed lastingly under positive pressure. The oral dispenser moreover has an atomization nozzle unit with at least one nozzle opening, and an outlet channel connecting the liquid reservoir to the atomization nozzle unit. An outlet valve in the outlet channel can adopt an opened state and a closed state. A mouthpiece is provided for inhaling the liquid atomized by the atomization nozzle unit. A refill opening is separate from the atomization nozzle unit and connected to the liquid reservoir, wherein the spring mechanism is loaded by liquid fed through the refill opening into the liquid reservoir.

### AREA OF APPLICATION AND PRIOR ART

The invention concerns an oral dispenser for discharging a liquid in atomized form, wherein the invention concerns in particular such a dispenser for discharging a liquid containing nicotine or cannabis.

Such dispensers are used in particular to break the habit of cigarette smoking or as an alternative to cigarette smoking. They usually have an elongate form similar to that of a cigarette, with a mouthpiece through which the user can inhale vaporized or atomized liquid. As well as nicotine or cannabis-containing liquid, other liquids with substances for breaking addiction can be discharged with a dispenser according to the invention. Furthermore, the dispenser according to the invention may also be used for prevention or treatment of acute or chronic respiratory tract disorders or diseases.

A known type of cigarette-like dispenser is based on an electric vaporization of in particular a nicotine-containing liquid. Such dispensers comprise a battery or accumulator which must be replaced or recharged.

Dispensers are also known in which the nicotine-containing liquid is atomized without electricity using a propellant gas. Document GB 2530980 A for example discloses such a dispenser. The disadvantage of these dispensers is that the user also inhales the propellant gas when using the dispenser.

### PROBLEM AND SOLUTION

It is the object of the invention to provide an alternative dispenser by means of which liquids, in particular nicotine-containing or cannabis-containing liquids, can be applied in atomized form by a user.

For this, an oral dispenser of the type cited initially is proposed, which has a liquid reservoir of variable internal volume for receiving the liquid prior to the discharge.

An oral dispenser in the sense of the invention is a dispenser in which atomized liquid is applied orally, wherein the target of the liquid may for example be the mouth cavity itself, the throat area, the larynx, the bronchi or also the lungs. Preferably, an oral dispenser according to the invention is configured as an inhalation dispenser in which it is provided that the user inhales during discharge of the liquid and thus draws the atomized liquid into the respiratory tract.

According to the preferred area of application, the oral dispenser is preferably filled with a nicotine-containing or cannabis-containing liquid, in order thereby to constitute a substitute for corresponding cigarettes. However, a dispenser according to the invention may also be used for other applications, for example as a dispenser for an asthma or COPD medicament, which are usually discharged by means of MDI dispensers.

The oral dispenser according to the invention has a spring mechanism by means of which, when the spring mechanism is loaded, the liquid in the liquid reservoir is permanently under positive pressure. This spring mechanism may be a conventional spring, for example a coil spring. A spring mechanism in the sense of the invention however may also comprise spring mechanisms which are able to store energy in other ways, by compression of a gas or by deformation of an elastically deformable material.

To achieve a smaller size of the dispenser, the liquid reservoir is preferably rather small. Preferably, it has a volume of maximum 10 ml, in particular preferably maximum 5 ml or 3 ml. This volume is the difference between its maximum liquid content and its residual liquid content which can no longer be discharged for structural reasons.

The pressure exerted by the spring mechanism on the liquid in the liquid reservoir serves to atomize the liquid. To this end, an oral dispenser according to the invention has an atomization nozzle unit. This has at least one very small nozzle opening, preferably several nozzle openings. The clear cross-section of the at least one nozzle opening is maximum 0.002 mm², preferably in particular maximum 0.001 mm² or maximum 0.0005 mm². The total area of the clear cross-sections of all nozzle openings amounts preferably to maximum 0.04 mm². The nozzle openings are preferably round.

Under sufficiently high pressure, the small nozzle size leads to a fine atomization of the liquid. It is preferably provided that the spring mechanism is configured such that in fully loaded state, with a completely filled liquid reservoir, it exerts a force which places the liquid in the liquid reservoir under a pressure of at least 4 bar, preferably at least 6 bar and in particular preferably at least 8 bar. It is furthermore preferably provided that, even following progressive evacuation of the liquid reservoir and hence with no longer fully loaded spring mechanism, the spring mechanism at all times can generate a pressure of at least 2 bar, preferably at least 3 bar in the liquid. This may be achieved in that a complete discharge of the spring mechanism is structurally prevented, so that for example the minimum volume of the liquid dispenser is reached while the spring device can still generate in the liquid a pressure of at least 2 bar, preferably at least 3 bar.

If several nozzle openings are provided, these are preferably provided as openings in a common nozzle plate which in particular may be fixed in a sleeve-like carrier made of plastic which is fixed in the oral dispenser, in particular in an inner component of the oral dispenser. The nozzle plate may for example be configured as a plastic plate, a metallic nozzle plate or as a silicon plate. It may be convex, so that the nozzle openings are oriented in diverging directions.

To prevent particles in the liquid being able to block the nozzle openings, a liquid filter, in particular a membrane filter, is provided in the outlet channel, preferably upstream of the at least one nozzle opening.

The liquid flow of the liquid discharged by the atomization nozzle unit is preferably comparatively small. With a liquid pressure of 4 bar present at the atomization nozzle unit and with the outlet valve fully opened, it amounts to preferably between 5 µl/second and 30 µl/second, in particular preferably between 10 µl/second and 20 µl/second. This small liquid flow has proved particularly suitable for the cigarette simulation provided by the oral dispenser. The subjective perception with such volume flows is similar to that of a cigarette.

The liquid reservoir is connected to the atomization nozzle unit via an outlet channel. An outlet valve is provided therein which can adopt an open and a closed state, in particular in that a valve orifice element with a valve orifice is moved between a position interrupting the outlet channel and a position opening the outlet channel. Preferably, the outlet valve is configured such that it also allows intermediate positions which lead to a reduced volume flow.

The liquid atomized by the atomization nozzle unit enters a mouthpiece which, when correctly used, is surrounded by the user’s mouth before the outlet valve is opened.

It is preferably provided that not only is the atomization nozzle unit configured for discharge into the mouthpiece, but in addition at least one air channel is provided through which air can flow into the mouthpiece when the user draws on the mouthpiece. The user thus inhales air and atomized liquid. Preferably, several air channels are provided, in particular preferably two, three, or four, which are distributed over the periphery on an inside of the mouthpiece. These may open into the mouthpiece via a part of the air channel which is configured as a ring. Accordingly, the inhaled air may, in the manner of a peripheral air curtain, prevent the atomized liquid from being deposited on the inside of the mouthpiece.

The at least one inlet into the air channel is preferably not provided directly on an outside of the mouthpiece, but on a portion of the housing which is widened relative thereto so as to reduce the risk of the user’s lips covering the inlet. The inlet may in particular be oriented radially or axially against an inhalation direction.

Although it is preferred that an oral dispenser according to the invention is configured as a refillable dispenser and to this end has a refill opening, it may also be desired to configure an oral dispenser of said type as a single-use dispenser in which there is no refilling of liquid into the liquid reservoir. In such a case, no refill opening is required. It is however considered advantageous if the oral dispenser is provided for refilling. For this, it has a refill opening which is separate from the atomization nozzle unit and connected to the liquid reservoir, wherein the spring mechanism can be loaded by liquid being fed through the refill opening into the liquid reservoir.

Said refill opening is preferably provided with an inlet valve which can be opened by external positive pressure and/or by mechanical pressure of a refill connector. As long as no refill connector of a refill dispenser is applied, the inlet valve is pressed into its closed position by internal pressure.

An oral dispenser according to the invention should preferably resemble a cigarette in its handling. It therefore preferably has an elongate form oriented in a main extent direction. In particular, its outer form is preferably completely or largely rotationally symmetrical, wherein largely rotational symmetrical means that the mouthpiece and/or an actuation element for controlling the outlet valve may constitute deviations from the rotational symmetry. An outer housing of the oral dispenser preferably has a maximum diameter orthogonal to the main extent direction of 16 mm, disregarding a movable actuation element protruding beyond the outer housing for the outlet valve. The length in the main extent direction, including the mouthpiece preferably provided at a proximal end, is preferably at least 80 mm.

In the case of said elongate form, the atomization nozzle unit and/or the mouthpiece is provided at the proximal end of the oral dispenser, which supports the function of cigarette substitution. The refill opening is preferably provided opposite this at a distal end of the oral dispenser. This is structurally simplest to implement.

The liquid reservoir has a variable volume. This means that the extraction of liquid does not lead to a reduced pressure in the liquid reservoir and need not be compensated by inflowing air. Instead, the volume encased by the walls of the liquid reservoir reduces during liquid discharge.

A preferred embodiment here provides that the liquid reservoir is delimited at least in portions by a wall of flexible form. The spring mechanism is configured to exert a force on the form-flexible wall oriented in the direction of reducing the size of the liquid reservoir. Embodiments are conceivable both in which the form-flexible wall itself constitutes the spring mechanism, and also those in which the spring mechanism is provided separately from the wall and exerts a force thereon. Combinations are also possible. A design with separate spring mechanism may be formed by a gas pressure chamber which is separated from the liquid reservoir by the flexible or a displaceable wall. In this gas pressure chamber, at minimum liquid reservoir volumes, for example a pressure of 3 bar may prevail which rises to 4 bar on filling of the liquid reservoir.

Preferably, the form-flexible wall is elastically deformable. For this, in particular it may be made of an elastomer. Such an elastic wall, when stretched by deformation, pressurizes the liquid in the liquid reservoir and therefore itself forms the spring mechanism or a part of the spring mechanism.

A preferred embodiment provides that the form-flexible wall is configured as an elastically expandable hose surrounding the liquid reservoir. When the liquid reservoir is filled, the hose is stretched tangentially.

It is particularly preferred if a central mandrel is provided which is surrounded by the elastic hose. With such a design, the liquid reservoir is provided at least in portions as a ring between an outer face of the central mandrel and an inside of the hose. The central mandrel here may in particular guarantee that, even during increasing evacuation of the liquid reservoir, a sufficiently high liquid pressure exists since it delimits the volume reduction of the liquid reservoir. As soon as the hose lies directly on the outside of the central mandrel, no further volume reduction is possible even if the hose is still under tangential stress.

In particular so that the hose can be mounted for such configurations, the central mandrel is formed conical at least in portions, preferably over the majority of its length. This allows the hose to be pushed on in the direction of the largest circumference of the central mandrel, wherein during application the tangential stress is caused or increased.

In addition, the central mandrel may be provided with a central liquid channel which extends in particular in the main extent direction of the oral dispenser. This liquid channel may form part of an outlet channel which connects the liquid reservoir to the atomization valve unit. Alternatively or additionally, the liquid channel may form a refill channel or part thereof which connects the refill opening to the liquid reservoir. The central mandrel may have a radial opening in order to connect the outlet channel and/or the refill channel to the ring region of the liquid reservoir outside the central mandrel.

A particularly advantageous design of the central mandrel provides that this is formed in two pieces, in particular with an inner mandrel component and an outer mandrel component. The two mandrel components, preferably the inner and outer mandrel components, are designed to enlarge their overall outer circumference during mounting by axial displacement relative to one another. Such a design allows advantageous mounting in which initially the one, in particular the outer mandrel component, is inserted in the hose, and wherein then the other, in particular the inner mandrel component, is inserted on or in the mandrel component previously inserted, so that the common circumference increases and the hose is thereby tangentially expanded.

In a design of the dispenser with an inlet valve at the refill opening, it may advantageously be provided that at least one valve face of the inlet valve is formed by the component which integrally also forms the elastic hose of the liquid reservoir.

An alternative variant of a normal dispenser according to the invention has, instead of a form-flexible wall, a displaceable wall which delimits the liquid reservoir at least in portions. This displaceable wall is preferably inserted in the manner of a piston in a cylindrical body and seals against the latter in the edge region. The spring mechanism here is configured to exert a force on the displaceable wall oriented in the direction of reducing the size of the liquid reservoir. The displaceable wall is displaceable in the main extent direction and in particular is preferably force-loaded by the spring mechanism in the direction of a proximal end of the oral dispenser. The spring device is preferably formed by a gas pressure chamber or a coil spring.

Like the central mandrel already mentioned, the oral dispenser may have a guide mandrel. Here, the liquid reservoir is provided at least in portions as a ring between an outer face of this guide mandrel and an inside of an outer wall. The guide mandrel guarantees a reproducible, tilt-free shift of the displaceable wall. It also allows provision of a central liquid channel inside the guide mandrel, thus extending through the liquid reservoir. This may constitute the outlet channel and/or the refill channel. In particular, preferably the liquid channel in the guide mandrel extends as a refill channel from a distal end to an end of the liquid reservoir pointing towards the proximal end, where it is connected to the liquid reservoir by means of a radial opening.

For use of the oral dispenser, the user surrounds the mouthpiece with his lips and on inhalation, the outlet valve opens so that the liquid is atomized by the atomization nozzle unit. Various structures are preferred for the design of the outlet valve.

In a particularly simple design, the outlet valve is configured as a manually actuatable outlet valve, i.e. operated by hand by the user. Particularly suitable here is a design with an actuation pusher on an outside of the oral dispenser, above all on a casing surface of the oral dispenser, by means of which the outlet valve can be brought into a closed and an open state. Manual pressing of the actuation pusher, in particular against the force of a return spring, causes the outlet valve to open. The actuation pusher is preferably linearly movable.

A design is also possible with a manually actuatable outlet valve which has a rotatable actuation element, by means of which the outlet valve can be brought into a closed and an open state, wherein the actuation element is preferably rotatable about a rotational axis parallel to the main extent direction. For example, a front and a rear portion of the elongate dispenser may be rotated relative to one another about this rotational axis in order to open the outlet valve which is configured as a rotary valve.

An alternative design of the outlet valve provides that the outlet valve is configured as a lip-actuatable outlet valve. The user can open this by compressing his lips. For this, an actuating face is provided on the mouthpiece or in the region of the mouthpiece which, on application of force by the user’s lips, causes the outlet valve to transfer from a closed to an open state.

This may be achieved for example in that the outlet valve has a pivotably movable actuating lever which is pivotable by application of force on the actuating face, and on which a valve orifice element is provided which can be transferred from a closed position to an open position by the pivot movement of the actuating lever.

A further alternative design of the outlet valve is configured as a breath-controlled outlet valve. For this, it has a vacuum chamber communicating with the mouthpiece. When the user sucks on the mouthpiece, this creates a reduced pressure in this chamber. The vacuum chamber is provided with a displaceable chamber wall which is either deformable in itself or displaceable as a whole, in particular preferably against the force of a return spring. Displacement of the chamber wall acts on a valve element, for example on a valve orifice element in the outlet channel, so that the reduced pressure in the vacuum chamber transfers the outlet valve from a closed state to an open state.

The outlet valve is preferably configured such that said types of switching of the valve, i.e. manual switching, switching via the lips, switching via suction on the mouthpiece, in each case also allow an incomplete opening of the valve. Preferably also only a slight manual actuation, a slight pressure of the lips and a slight suction on the mouthpiece cause a correspondingly reduced discharge of atomized liquid. The user may thus control the amount of flow of atomized liquid. In particular, this can be achieved by a valve orifice element which is moved away from the outlet channel to different extents depending on actuation, and thus acts to a variable extent as a limiting choke.

The structure of the oral dispenser preferably provides that this has an outer housing which also forms the mouthpiece, and within which preferably at least one in a component is arranged. In a preferred embodiment, the outer housing may be formed by two half-shells, between which the at least one inner component is provided. A design is preferred with an inner component having a valve recess in which the valve orifice element of the outlet valve is movably mounted, and/or which in portions forms the outlet channel and/or which forms the guide mandrel or the central mandrel.

According to a second aspect of the invention, the invention furthermore also concerns an oral dispenser for discharging a liquid containing nicotine or cannabis, wherein this may comprise some or all of the above-mentioned features individually or in combination or in partial combination.

The oral dispenser according to the second aspect of the invention has a liquid reservoir for receiving the nicotine- or cannabis-containing liquid prior to the discharge. On delivery of the liquid reservoir, this is preferably already filled with the corresponding liquid.

The liquid reservoir is configured as a pressure reservoir. This means that the liquid is stored in the liquid reservoir under pressure. The pressurization may for example be achieved by a propellant gas or, in the above-described fashion, by a spring mechanism.

The oral dispenser has an atomization nozzle unit. This may be designed in various ways. In the simplest case, it has an eddy chamber by means of which the atomization is achieved. It may however also be provided with one or more very fine nozzle openings in the manner described above. The individual nozzle openings here preferably have a clear cross-section of maximum 0.002 mm². The entirety of nozzles preferably has a total of clear cross-section of maximum 0.04 mm².

The oral dispenser has an outlet channel which connects the liquid reservoir to the atomization nozzle unit. An outlet valve is provided in this outlet channel which can adopt an open and a closed state. When the outlet valve is opened, the liquid flows out of the pressure reservoir in the direction of the nozzle unit, where it is discharged in atomized form through a mouthpiece of the dispenser. The outlet valve may for example be provided in the manner of a movable valve orifice element which is provided with an orifice. If the valve orifice element and hence the orifice are moved upstream and downstream relative to the outlet channel, the flow may thereby be terminated. If the orifice is shifted by displacement of the valve orifice element so that it aligns with the outlet channel, the liquid can flow through.

A feature of this second aspect of the invention is that the outlet valve is configured as a mechanically lip-actuatable outlet valve. For this, an actuating face is provided on the mouthpiece or in the region of the mouthpiece which, on application of force from the user’s lips, mechanically causes the outlet valve to transfer from a closed to an open state.

This manner of actuation is highly intuitive and therefore particularly suitable for a nicotine or cannabis dispenser which is used very frequently, and in which it is therefore desired that the discharge should not be associated with difficult operation, but instead should be possible as intuitively as possible. It has been found that lip actuation particularly advantageously allows the user to temporally match the inhalation with the discharge of the atomized liquid caused by means of the lips. It is advantageous that the oral dispenser has an air inlet channel, in particular at the mouthpiece, through which the user can draw in air even before the beginning of discharge, in order then during this inhalation to begin discharge by the force applied by means of the lips.

Said actuating face is preferably less than 20 mm, in particular preferably less than 15 mm, away from a distal end of the mouthpiece. When the user takes the mouthpiece in his mouth and surrounds it with his lips, one of his lips, preferably the top lip, rests on this actuating face. The actuating face is in particular depressible radially, i.e. in the direction of the mouthpiece. The user may thus open the valve by compressing his lips. Return to the closed starting state preferably takes place by spring force. In particular, preferably the valve is configured to also allow partial opening. This allows the user to regulate the through-flow quantity by the application of force by means of his lips, and thereby determine the quantity of nicotine or cannabis in a similar fashion to sucking with variable strength on a cigarette.

In the manner described above, the liquid reservoir may be configured with a variable inner volume. It reduces in size as liquid is discharged. If the dispenser is configured as a single-use dispenser, it is discarded after evacuation of the liquid reservoir. However, in the manner described above, it may also be configured as a refillable dispenser. In this case, here again it is preferably provided that the dispenser has a refill opening, separate from the atomization nozzle unit, which is connected to the liquid reservoir, wherein the spring mechanism is loaded by liquid being fed through the refill opening to the liquid reservoir.

The invention concerns not only the oral dispenser itself but also an inhalation system with such an oral dispenser, which additionally comprises a refill dispenser in which pressurized liquid or pressurizible liquid is stored and which is configured for coupling to the refill opening of the oral dispenser. Preferably, the oral dispenser is filled with pressurized liquid without propellant, wherein the liquid can be extracted through a switchable discharge valve. This valve is preferably provided with a refill connector which is adapted to the refill opening of the oral dispenser and can be inserted therein. The insertion opens the discharge valve of the refill dispenser and the inlet valve of the oral dispenser, so that the liquid can flow into the liquid reservoir of the oral dispenser and there reload the spring device.

### BRIEF DESCRIPTION OF THE DRAWINGS

Further advantages and aspects of the invention arise from the claims and the following description of preferred exemplary embodiments of the invention which are explained below with reference to the figures.

FIG. 1 shows an oral dispenser according to a first preferred design in perspective view.

FIGS. 1A to 1C show the oral dispenser of FIG. 1 in other perspectives.

FIGS. 2A and 2B show the oral dispenser from FIG. 1 in two sectional views.

FIG. 2C shows an enlarged illustration of the atomization nozzle unit of the oral dispenser.

FIGS. 3A and 3B illustrate the loading of the oral dispenser.

FIGS. 3C and 3D illustrate the use of the oral dispenser.

FIG. 4 shows an oral dispenser according to a second proposed design in perspective view.

FIGS. 5A and 5B show the oral dispenser from FIG. 4 in two sectional views.

FIGS. 6 and 7 show two alternative concepts for implementing an outlet valve which may be used in both designs of an oral dispenser according to FIGS. 1 and 4.

FIGS. 8A to 8C show an alternative form of a central mandrel for the oral dispenser according to FIGS. 1 to 3D.

### DETAILED DESCRIPTION OF THE EXEMPLARY EMBODIMENTS

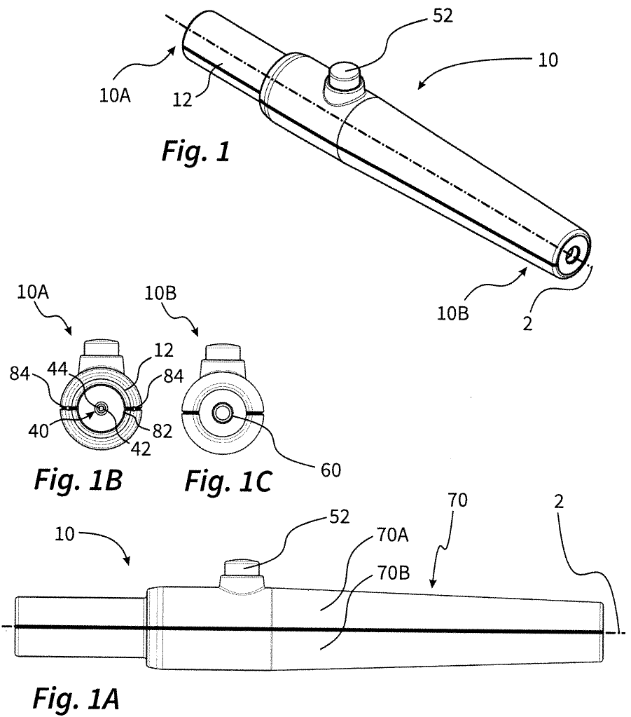

FIGS. 1 to 3D show a first embodiment of an oral dispenser according to the invention.

FIG. 1 shows the oral dispenser 10 in an overall perspective illustration. The oral dispenser 10 has an elongate housing oriented in a main extent direction 2, at the proximal end 10A of which a mouthpiece 12 is provided. On its casing surface in this exemplary embodiment, an actuating pusher 52 is provided, by means of which a further outlet valve 50 (to be explained below) can be opened.

With reference now to FIGS. 1A to 1C, it is clear that at the proximal end in the mouthpiece 12, an atomization nozzle unit 40 is arranged which has a nozzle plate 42 with a plurality of nozzle openings 44. On the outer edge of the approximately cylindrical mouthpiece 12, a ring channel 82 is also provided, through which air can be drawn in through the mouthpiece 12. To this end, the air is drawn in through inlets 84.

A refill opening 60 is provided at the opposite distal end 10B of the oral dispenser 10.

As shown in FIG. 1A, an outer housing 70 of the oral dispenser 10 comprises two half-shells 70A, 70B. These half-shells contain a plurality of components (to be described below) which serve for mounting and for targeted atomization of liquid.

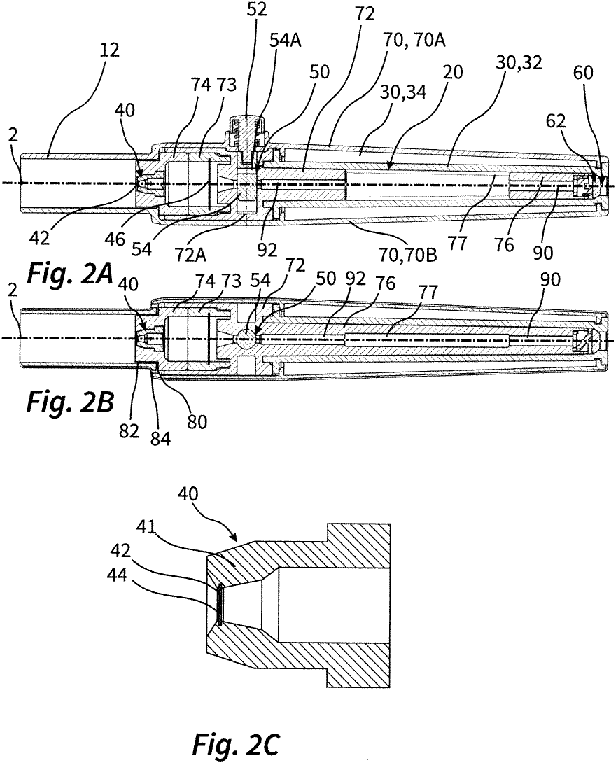

FIGS. 2A and 2B show the oral dispenser 10 in sectional illustration, wherein the section planes are offset by 90° relative to one another.

It is clear from the figures that three inner components 72, 73, 74 are provided inside the outer housing 70. The inner component 72, at its right-hand end in FIG. 2A, forms a central mandrel 76 which extends almost up to the distal end 10B of the oral dispenser 10. A hose-like component 32 of elastomer is pushed over this central mandrel 76. A space between an outside of the central mandrel 76 and an inside of this hose 32 forms the liquid reservoir 20. In the illustrations in FIGS. 2A and 2B, this liquid reservoir is not filled. The liquid reservoir is connected by radial openings 77 to a liquid channel 90, 92 which extends through the central mandrel 76 in the main extent direction 2.

The left-hand part 92 of this liquid channel in FIGS. 2A and 2B forms part of an outlet channel 92. Starting from the liquid reservoir 20, this outlet channel 92 leads to the outlet valve 50. In the design in FIGS. 1 to 3D, in addition to the actuating pusher 52, a valve orifice element 54 with an orifice 54A is attached this outlet valve 50 so as to be movable inside a valve recess 72A. The actuating pusher 52 is pressed into the position of FIG. 2A by a return spring. When pressed down against the force of the return spring, the orifice 54A can be brought into a position aligning with the outlet channel 92 so that the liquid flows through the valve orifice element 54 to the right-hand side. On the right-hand side and hence downstream of the outlet valve 50, firstly a liquid filter 46 is provided, which is configured as a membrane filter, inserted in the inner component 73 and preferably encapsulated thereby. The liquid flowing through the outlet valve 50 is thus freed of any contaminants which may be present and would otherwise be able to disrupt the function of the atomization nozzle unit 40.

The above-mentioned atomization nozzle unit 40 is situated downstream of the membrane filter 46. Its main component is a thin nozzle plate, for example made of silicon, which is punctured by a plurality of nozzle openings 44. The nozzle plate 42 is fixed in a sleeve-like plastic carrier 43 which in turn is inserted in a corresponding recess of the inner component 74.

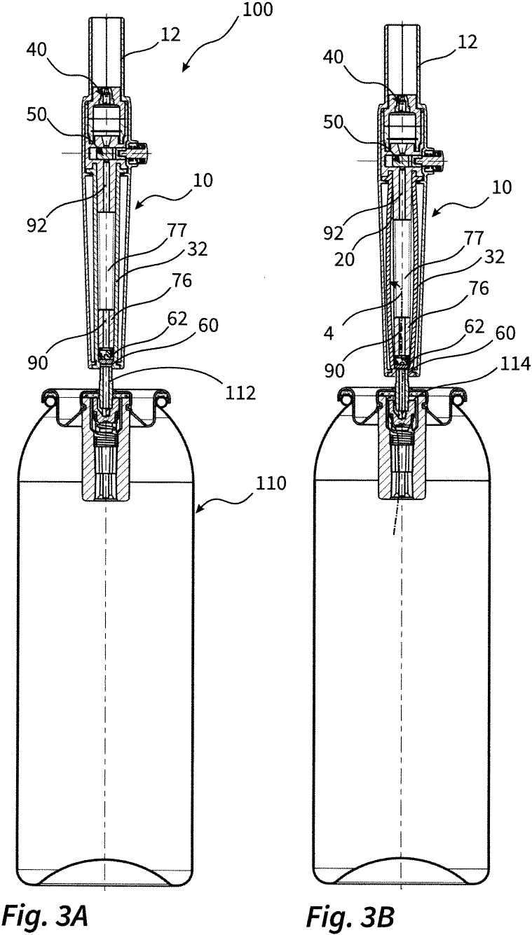

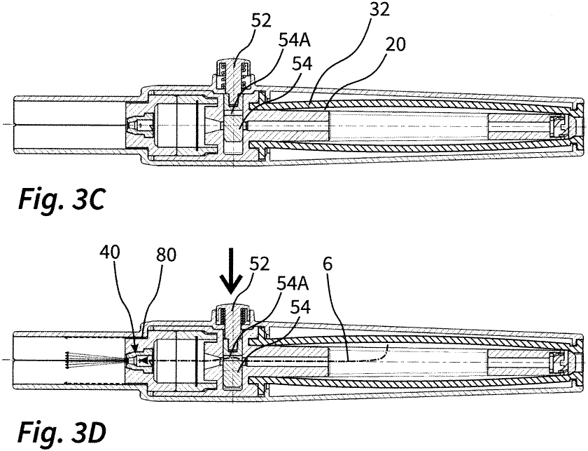

Upstream of the described liquid reservoir 20 is the refill channel 90, as a channel provided in the central mandrel 76 and extending to the refill opening 60. The liquid reservoir 20 is however normally isolated against the external environment since an inlet valve 62 is provided in the form of an overpressure valve, which is pressed into its closed position under the positive pressure usually prevailing in the oral dispenser 10. For this, a valve body of the inlet valve 62 is pressed against an elastically deformable ring region which is formed by the component of the hose 32. This is explained below with reference to FIG. 3B.

As evident from FIG. 2B and also FIG. 1C, and as has already been mentioned in this context, the oral dispenser 10 has two air channels 80 which each open into the above-mentioned ring channel 82, which itself opens into the mouthpiece 12.

FIG. 2C shows an enlarged view of the atomization nozzle unit 40. It shows the sleeve component 41 surrounding a passage, in which the nozzle plate 42 with nozzle openings 44 is provided. The nozzle openings each have a clear cross-section of 100 µm². In the present exemplary embodiment, 25 such nozzle openings 44 are provided.

FIGS. 3A and 3B illustrate the process of filling the oral dispenser 10. To fill the oral dispenser 10, a refill dispenser 110 is provided which, together with the oral dispenser 10, forms an inhalation system 100.

The refill dispenser 110 has a pressure reservoir with an outlet valve 114 at its upper end which can be opened by means of a valve connector. The valve connector forms a refill connector 112 for insertion into the refill opening 60 of the oral dispenser 10. If the oral dispenser 10, as illustrated in FIG. 3B, is pressed down on the refill opening 60 after application of the refill connector 112, the outlet valve 114 of the refill dispenser 110 is opened, and by force application by means of the refill connector 112, the inlet valve 62 of the oral dispenser 10 is also opened.

Since a pressure of 8 bar prevails in the refill dispenser 110, liquid can pass into the refill channel 90 through the refill connector 112 and into the liquid reservoir 20 of the oral dispenser. The liquid path 4 illustrates this.

There, under the pressure prevailing in the refill dispenser 110, the hose 32 is expanded and thereby elastically deformed such that, after removal of the oral dispenser 10 from the refill dispenser 110 and the resulting closure of the inlet valve 62, it exerts a pressure of around 4 bar on the liquid present in the liquid reservoir 20.

FIG. 3C shows the now loaded oral dispenser 10 with expanded hose 32.

If now, in the manner shown in FIG. 3D, the outlet valve 50 is opened by depressing the actuating pusher 52, the liquid flows out of the liquid reservoir 20 along the liquid path 6 through the orifice 54A of the valve orifice element 50 to the atomization nozzle unit 40, which performs the atomization as illustrated with the dotted arrows in FIG. 3D. At the same time, the user sucks on the mouthpiece 12, whereby an external air jacket is generated via the air channels 80 in the mouthpiece 12 and is inhaled by the user together with the atomized liquid.

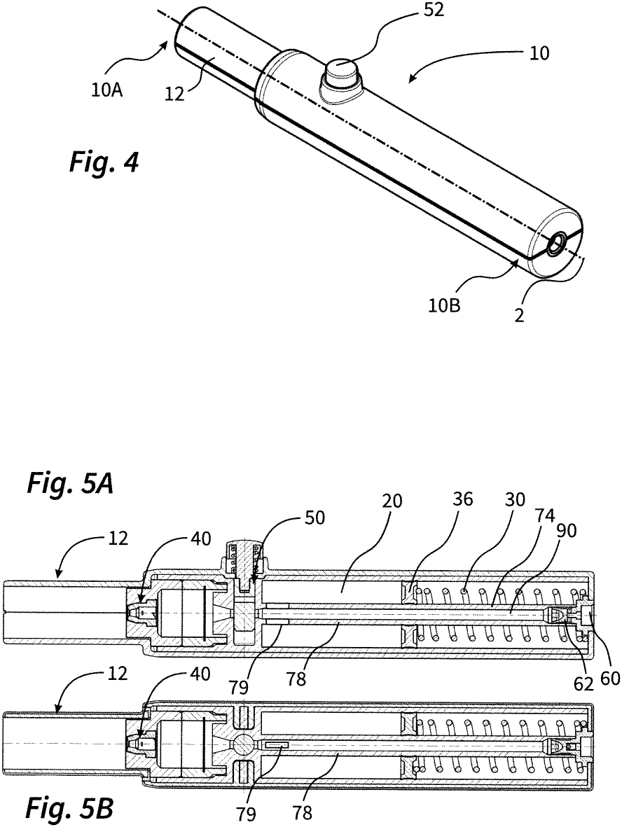

FIGS. 4, 5A and 5B illustrate an alternative structure of the oral dispenser 10. The main difference between the structure of the preceding figures and this design is that in this design, the liquid reservoir 20 is delimited by a displaceable wall in the manner of a piston 36. This is guided by a central guide mandrel 78 and permanently force-loaded in the direction of the mouthpiece 12 by a spring mechanism 30 formed as a coil spring. When the liquid reservoir 20 is filled, because of the spring mechanism 30, the liquid stands under a pressure which is sufficiently high for atomization to take place at the atomization nozzle unit 40 in the fashion described above. As the discharge of the liquid from the liquid reservoir 20 progresses, the piston 36 is moved further in the direction of the mouthpiece.

When the refill dispenser 110 is applied in this design, the liquid reservoir 20 is here refilled, while at the same time the piston 36 is deflected further against the force of the spring mechanism 30. For this, the liquid flows from the refill opening 60 through the inlet valve 62 (here formed as an overpressure valve) into the refill channel 90 and through radial openings 74 in the guide mandrel 78 into the liquid reservoir 20.

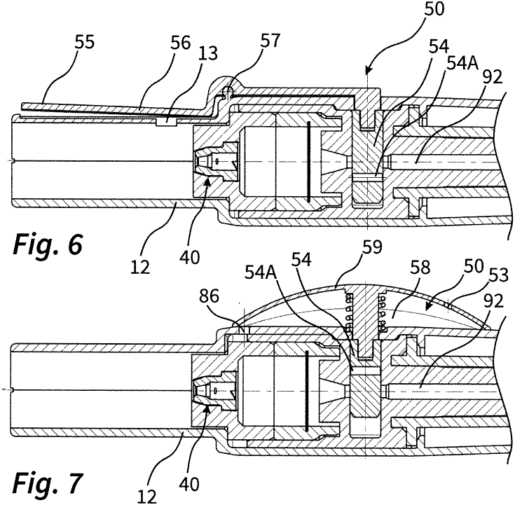

FIGS. 6 and 7 show alternative designs of the outlet valve which may be implemented equally well in both preceding embodiments of the liquid reservoir.

With the design according to FIG. 6, lip actuation of the outlet valve 50 is provided. For this, an actuation lever 56 is provided which is pivotable about a pivot axis 57, wherein the proximal end of said lever has an actuating face 55 which is pressed down in correct use by compression of the user’s lips. The orifice 54A of the valve orifice element 54 is provided on the opposite leg of the actuating lever 56. When the actuating face 55 is pressed down, the orifice 54A is raised so that it aligns with the adjoining parts of the outlet channel 92, and hence liquid passes from the liquid reservoir 20 to the atomization nozzle unit 40.

An air inlet opening 13 is provided on the mouthpiece 12. This allows the user to surround the mouthpiece and actuating face 55 with his lips and then draw in air through the air inlet opening 13 even before actuation of the actuating face 55. During this process, the discharge of the nicotine- or cannabis-containing liquid can be started by compression of the user’s lips and hence depression of the actuating face 55. By judicious application of force, the user is also able to align the orifice 54A only partially with the outlet channel 92 and thereby achieve only a low discharge of liquid. In a similar fashion to a cigarette, the user can thus control the discharge according to his wish.

The design according to FIG. 7 is a design with breath actuation of the outlet valve 50. When the user sucks on the mouthpiece 12, this not only serves to draw in air through the already described air channels 80, but also to extract air through the additional air channel 86 from a vacuum chamber 58 of the outlet valve 50, and the flexible chamber wall 59 of the vacuum chamber 58 therefore presses the valve orifice element 54 downward. In this way again, the orifice 54A in the valve orifice element 54 comes into communication with adjoining parts of the outlet channel 92 and liquid flows to the atomization nozzle unit 40.

In the example of FIG. 7, the vacuum chamber is connected to the environment via an opening 53. The flow resistance through the opening is greater than the flow resistance through the channel 86. Thus the vacuum is created in the vacuum chamber 58. An additional air channel corresponding to the above-described air channels 80 is not necessary with this design, since air can reach the mouthpiece through the opening 53 and the air channel 86.

In alternative designs, the opening 53 may also be omitted and instead the described air channels 80 provided. With suitable design of the respective choke effects of the openings, a vacuum can then be created in the vacuum chamber 59.

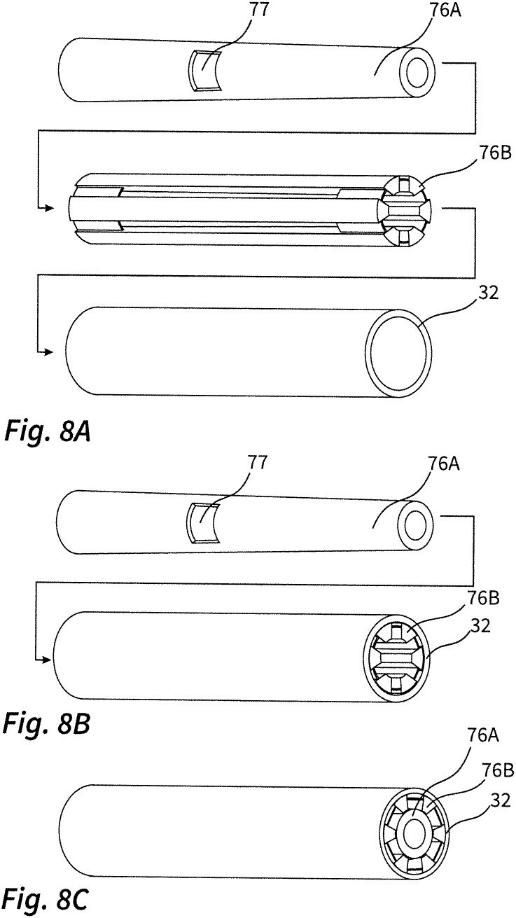

FIGS. 8A to 8C show a particular design of the central mandrel 76 and associated mounting. The central mandrel 76 is here divided into two mandrel components 76A, 76B. The inner mandrel component 76A has a slightly conical form. The outer mandrel component 76B has a plurality of ribs which are distributed circumferentially and connected together by thin connecting webs. The ribs themselves are formed conical on the inside corresponding to the conicity of the inner mandrel component 76A.

To mount the two-piece central mandrel in a hose 32, it is provided according to FIG. 8B that firstly the outer mandrel component 76B is inserted in the hose 32. This is no problem since, in the starting state of FIG. 8A, the outer diameter of the outer mandrel component 76B is smaller than the inner diameter of the hose 32.

Then the assembly of hose 32 and outer mandrel component 76B is pushed onto the conical inner mandrel component 76A. The outer mandrel component 76B thereby expands and with it the hose 32, so that the latter is placed under the desired pretension.

This structure of the liquid reservoir constitutes a version which is improved with respect to installation in comparison with the design of the central mandrel 76 of FIGS. 1 to 3D.

1. An oral dispenser for discharging a liquid in atomized form, comprising:
a liquid reservoir of variable internal volume for storing the liquid prior to discharge;
a spring mechanism by which, when the spring mechanism is loaded, the liquid in the liquid reservoir is permanently under positive pressure;
an atomization nozzle unit with at least one nozzle opening with a maximum cross-section of 0.002 mm²;
an outlet channel connecting the liquid reservoir to the atomization nozzle unit;
an outlet valve provided in the outlet channel which can adopt an open state and a closed state;
a mouthpiece for inhalation of the liquid atomized by the atomization nozzle unit; and
a refill opening separate from the atomization nozzle unit and connected to the liquid reservoir, wherein the spring mechanism is loaded by liquid fed through the refill opening into the liquid reservoir.

2. The oral dispenser as claimed in claim 1, including
a nicotine-containing liquid in the liquid reservoir or
a cannabis-containing liquid in the liquid reservoir.

3. The oral dispenser as claimed in claim 1, wherein
the spring mechanism is configured such that in a fully loaded state with the liquid reservoir completely filled, the spring mechanism exerts a force which places the liquid in the liquid reservoir under a pressure of at least 4 bar.

4. The oral dispenser as claimed in claim 1, wherein
the oral dispenser has an elongate form oriented in a main extent direction.

5. The oral dispenser as claimed in claim 1, wherein
the liquid reservoir is delimited at least in portions by a wall having a flexible form, and
the spring mechanism is configured for exerting a force on the form-flexible wall to reduce a size of the liquid reservoir.

6. The oral dispenser as claimed in claim 5, wherein
the form-flexible wall is configured as an elastically expandable hose surrounding the liquid reservoir.

7. The oral dispenser as claimed in claim 6, wherein
the oral dispenser has a central mandrel surrounded by the elastic hose, and the liquid reservoir is provided at least partially as a ring between an outer face of the central mandrel and an inside of the hose.

8. The oral dispenser as claimed in claim 1,
the liquid reservoir is delimited at least in portions by a displaceable wall, and
the spring mechanism is configured to exert a force on the displaceable wall, the force being oriented in a direction to reduce the size of the liquid reservoir.

9. The oral dispenser as claimed in claim 1, wherein the at least one nozzle opening comprises
a plurality of nozzle openings configured as openings in a common nozzle plate.

10. The oral dispenser as claimed in claim 1, wherein
the outlet valve is configured as a manually actuatable outlet valve.

11. The oral dispenser as claimed in claim 1, wherein
the outlet valve is configured as a lip-actuatable outlet valve and has an actuating face on the mouthpiece or in the region of the mouthpiece, the actuating face, when force-loaded by a user’s lips, causing the outlet valve to transfer from the closed to the open state.

12. The oral dispenser as claimed in claim 1, wherein
the outlet valve is formed as a breath-controlled outlet valve and has a vacuum chamber in communication with the mouthpiece and provided with a displaceable chamber wall, the displaceable chamber wall transferring the outlet valve from the closed to the open state by vacuum-induced displacement.

13. The oral dispenser as claimed in claim 1, with at least one of the following further features:
the refill opening is provided with an inlet valve openable by external positive pressure and/or by mechanical pressure of a refill connector and pressable into a closed position by internal pressure; and/or
the oral dispenser has an outer housing; and/or
the oral dispenser has an inner component having a valve recess in which a valve orifice element of the outlet valve is movably mounted, and/or the inner component in portions forming the outlet channel, and/or the inner component forming a guide mandrel or a central mandrel; and/or
the liquid reservoir has a maximum volume of 10 ml; and/or
a liquid filter is arranged in the outlet channel upstream of the atomization nozzle unit.

14. The oral dispenser as claimed in claim 1, further comprising
at least one air channel through which air flows into the mouthpiece in order to be inhaled with the atomized liquid.

15. An inhalation system comprising:
an oral dispenser for discharging a liquid in atomized form, the oral dispenser comprising:
a liquid reservoir of variable internal volume for storing the liquid prior to discharge;
a spring mechanism by which, the spring mechanism is loaded, the liquid in the liquid reservoir is permanently under positive pressure;
an atomization nozzle unit with at least one nozzle opening with a maximum cross-section of 0.002 mm²;
an outlet channel connecting the liquid reservoir to the atomization nozzle unit;
an outlet valve provided in the outlet channel which can adopt an open state and a closed state;
a mouthpiece for inhalation of the liquid atomized by the atomization nozzle unit; and
a refill opening separate from the atomization nozzle unit and connected to the liquid reservoir, wherein the spring mechanism is loaded by liquid fed through the refill opening into the liquid reservoir;
a refill dispenser containing pressurized liquid or pressurizable liquid and configured for coupling to the refill opening of the oral dispenser.

16. An oral dispenser for discharging a liquid containing nicotine or cannabis in atomized form, comprising:
a liquid reservoir for storing the nicotine- or cannabis-containing liquid prior to discharge,
the liquid reservoir being formed as a pressure reservoir storing the liquid under positive pressure;
an atomization nozzle unit;
an outlet channel connecting the liquid reservoir to the atomization nozzle unit;
an outlet valve provided in the outlet channel and having an open state and a closed state; and
a mouthpiece for inhalation of the liquid atomized by the atomization nozzle unit,
wherein the outlet valve is configured as a mechanically lip-actuatable outlet valve and has an actuating face on the mouthpiece or in the region of the mouthpiece, the actuating face, under force-loading from a user’s lips, causing the outlet valve to transfer from the closed to the open state.

17. The oral dispenser as claimed in claim 16, wherein
the outlet valve has a pivotingly movable actuating lever, the actuating lever being pivotable by force-loading of the actuating face and the outlet valve including a valve orifice element movable from a closed position to an open position by the pivot movement of the actuating lever.

18. The oral dispenser as claimed in claim 16, wherein
the oral dispenser has an air inlet channel through which ambient air is drawn in.

19. The oral dispenser as claimed in claim 16,with at least one the following further features:
the liquid reservoir has a variable inner volume; and/or
the oral dispenser has a spring mechanism, wherein when the spring mechanism is loaded, the liquid in the liquid reservoir is permanently under positive pressure; and/or
the atomization nozzle unit has at least one nozzle opening with a maximum cross-section of 0.002 mm²; and/or
the oral dispenser has a refill opening separate from the atomization nozzle unit and connected to the liquid reservoir, wherein the spring mechanism is loaded by liquid being fed through the refill opening into the liquid reservoir; and/or
the liquid reservoir is filled with a nicotine- or cannabis-containing liquid.

20. The oral dispenser as claimed in claim 1, wherein the at least one nozzle opening comprises a plurality of nozzle openings having cross-sections in total amounting to a maximum of 0.04 mm².

21. The oral dispenser as claimed in claim 3, wherein the atomization nozzle unit is configured such that with an internal liquid pressure of 4 bar, a liquid flow between 5 µl/second and 30 µl/second is dispensed by the atomization nozzle unit.

22. The oral dispenser as claimed in claim 4, wherein the refill opening is arranged at a distal end of the oral dispenser.
